(19) United States
(12) Patent Application Publication
(10) Pub. No.: US 2011/0135746 A1
(43) Pub. Date: Jun. 9, 2011

(54) Protective lip balm composition

(76) Inventors: Janice L. Triplett, Sutter, CA (US); David C. Triplett, JR., Sutter, CA (US)

(21) Appl. No.: 12/653,160

(22) Filed: Dec. 9, 2009

(51) Int. Cl. A61K 35/24

(52) U.S. Cl. 424/537

(57) ABSTRACT

Lip balm formulations are disclosed that comprise a combination of anti-bacterial, anti-viral, and anti-fungal substances, including: bees wax, cocoa butter, Shea butter, Jojoba oil, Naroli oil, myrrh, tea tree oil, lavender oil, Blue Moroccan chamomile, and vitamin E and, optionally, Manuca honey and aloe vera either singularly or together with the other ingredients.

### BACKGROUND OF THE INVENTION

1. Field of the Invention

This invention pertains generally to a protective composition for application to lips and other suitable skin locations, and more particularly to a topical composition having desirable moisturizing, antibacterial, antifungal, and antiviral properties.

2. Description of Related Art

Various formulations exist for lip balms or protective coating for skin. A few exemplary patents, of the many examples for lip balm patents, include: U.S. Pat. No. 4,551,332 discloses a Vitamin E composition for use as a dermatological coating.

U.S. Pat. No. 5,503,825 presents a lip balm composition comprising aloe vera and salt as a large portion of its formulation.

U.S. Pat. No. 6,136,301 discloses a lipid mix for lip protection. Included in the composition are cholesteryl esters and sphingolipids of various formulas. Additionally, cosmetic and pharmaceutical ingredients for topical applications are incorporated into the product.

### BRIEF SUMMARY OF THE INVENTION

An object of the present invention is to provide a lip balm containing natural oils and herbs which have been, individually, utilized in various other settings (other than in lip balm and not in a mixture similar to the subject composition) as antibacterial, antifungal, and antiviral agents. The subject invention formulation offers these antibacterial, antifungal, and antiviral properties in a single product for convenient application. The subject product works in conjunction with natural body defenses to improve protection against bacterial, fungal, and viral infections. The principle in the design of the subject lip balm is that the active presence of known (from other settings than a lip balm component) and recognized natural products with anti-bacterial, anti-viral, and anti-fungal properties when placed on the lips provides a significant reduction in the amount of harmful contaminants entering the body via the mouth.

Another object of the present invention is to furnish a lip balm formulation comprising various anti-bacterial, anti-viral, and anti-fungal substances, including: bees wax, cocoa butter, Shea butter, Jojoba oil, Naroli oil, myrrh, tea tree oil, lavender oil, Blue Moroccan chamomile, and vitamin E.

A further object of the present invention is to supply a lip balm formulation comprising various anti-bacterial, anti-viral, and anti-fungal substances, including: bees wax, cocoa butter, Shea butter, Jojoba oil, Naroli oil, myrrh, tea tree oil, lavender oil, Blue Moroccan chamomile, and vitamin E and, optionally, Manuca honey and aloe vera.

Disclosed is a topical lip balm material that contains materials that may be used in connection with bacterial, viral, and fungal infections by being applied to a user's lips, thereby contacting bacterial, viral, and fungal organisms as they enter the mouth of a user. The subject lip balm formulation comprises various anti-bacterial, anti-viral, and anti-fungal substances, including: bees wax, cocoa butter, Shea butter, Jojoba oil, Naroli oil, myrrh, tea tree oil, lavender oil, Blue Moroccan chamomile, and vitamin E and, optionally, Manuca honey and aloe vera.

Further objects and aspects of the invention will be brought out in the following portions of the specification, wherein the detailed description is for the purpose of fully disclosing preferred embodiments of the invention without placing limitations thereon.

### DETAILED DESCRIPTION OF THE INVENTION

Disclosed is an anti-bacterial, anti-viral, anti-fungal topical lip balm material that may be used in connection with bacterial, viral, and fungal infections by being applied to a user's lips, thereby contacting bacterial, viral, and fungal organisms as they enter the mouth of a user. Preferably, the subject lip balm formulation comprises a combination of anti-bacterial, anti-viral, and anti-fungal substances. Included in the preferred formulation is: bees wax, cocoa butter, Shea butter, Jojoba oil, Naroli oil, myrrh, tea tree oil, lavender oil, Blue Moroccan chamomile, and vitamin E. Optionally, Manuca honey and aloe vera are also included either singularly or together with the other ingredients noted immediately above.

The subject invention comprises a general formulation disclosed in TABLE 1, with optional ingredients listed in TABLE 2. As is seen in the presented preferred % ranges (by volume ranges), the bulk (majority of the composition) of the formulations comprises bees wax, cocoa butter, Shea butter, and Jojoba oil. In combination with the bulk substances, the listed additional ingredients produce desirable antibacterial, antifungal, and antiviral properties.

TABLE 1
Ingredients

| Ingredient | Preferred % Range by Volume |
|---|---|
| 1) Bees Wax | ~10 to ~25% |
| 2) Cocoa Butter | ~5 to ~15% |
| 3) Shea Butter | ~30 to ~50% |
| 4) Jojoba Oil | ~20 to ~35% |
| 5) Naroli Oil | ~0.05 to ~0.5% |
| 6) Myrrh | ~0.05 to ~0.5% |
| 7) Tea Tree Oil | ~0.05 to ~0.5% |
| 8) Lavender Oil | ~0.05 to ~1% |
| 9) Blue Moroccan Chamomile | ~0.05 to ~0.5% |
| 10) Vitamin E | ~1 to ~7% |

TABLE 2
Optional Ingredients

| Optional Ingredients | Preferred % Range by Volume |
|---|---|
| 11) Manuca Honey | ~0.1 to ~8% |
| 12) Aloe Vera | ~0.1 to ~8% |

### EXAMPLES

### Example 1

A first preferred embodiment of the subject formulation is related in TABLE 3 (Formulation #9).

TABLE 3
Formulation #9

| Ingredient | % by Volume |
|---|---|
| 1) Bees Wax | 20% |
| 2) Cocoa Butter | 7% |
| 3) Shea Butter | 38% |
| 4) Jojoba Oil | 32% |
| 5) Naroli Oil | 0.2% |
| 6) Myrrh | 0.1% |
| 7) Tea Tree Oil | 0.2% |
| 8) Lavender Oil | 0.2% |
| 9) Blue Moroccan Chamomile | 0.1% |
| 10) Vitamin E | 2% |

The ingredients listed in TABLE 3 (Formulation #9) are added together and blended at a suitable temperature to produce a semi-fluid mixture and then solidified, by cooling, in a sealable cylinder to generate a first embodiment of the subject product.

### Example 2

A second preferred embodiment of the subject formulation is related in TABLE 4 (Formulation #12).

TABLE 4
Formulation #12

| Ingredient | % by Volume |
|---|---|
| 1) Bees Wax | 13% |
| 2) Cocoa Butter | 11% |
| 3) Shea Butter | 45% |
| 4) Jojoba Oil | 20% |
| 5) Naroli Oil | 0.3% |
| 6) Myrrh | 0.3% |
| 7) Tea Tree Oil | 0.2% |
| 8) Lavender Oil | 0.6% |
| 9) Blue Moroccan Chamomile | 0.3% |
| 10) Vitamin E | 7% |
| 11) Manuca Honey | 2% |

The ingredients listed in TABLE 4 (Formulation #12) are added together and blended at a suitable temperature to produce a semi-fluid mixture and then solidified, by cooling, in a sealable cylinder to generate a second embodiment of the subject product.

### Example 3

A second preferred embodiment of the subject formulation is related in TABLE 5 (Formulation #16).

TABLE 5
Formulation #16

| Ingredient | % by Volume |
|---|---|
| 1) Bees Wax | 19% |
| 2) Cocoa Butter | 10% |
| 3) Shea Butter | 32% |
| 4) Jojoba Oil | 32% |
| 5) Naroli Oil | 0.2% |
| 6) Myrrh | 0.2% |
| 7) Tea Tree Oil | 0.4% |
| 8) Lavender Oil | 0.8% |
| 9) Blue Moroccan Chamomile | 0.2% |
| 10) Vitamin E | 3% |
| 11) Manuca Honey | 1% |
| 12) Aloe Vera | 1% |

The ingredients listed in Table 5 (Formulation #16) are added together and blended at a suitable temperature to produce a semi-fluid mixture and then solidified, by cooling, in a sealable cylinder to generate a third embodiment of the subject product.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

What is claimed is:

1. A lip balm composition comprising at least 65%, by volume, a combination of bees wax, cocoa butter, Shea butter, and Jojoba oil.

2. A lip balm composition according to claim 1, further comprising Naroli oil, myrrh, tea tree oil, lavender oil, Blue Moroccan chamomile, and vitamin E.

3. A lip balm composition according to claim 2, further comprising an ingredient selected from a group consisting of Manuca honey and aloe vera.

4. A lip balm composition according to claim 2, further comprising Manuca honey and aloe vera.

5. A lip balm composition comprising a combination of ˜10% to ˜25% bees wax, ˜5% to ˜15% cocoa butter, ˜30% to ˜50% Shea butter, and ˜20% to ˜35% Jojoba oil, wherein all percentages are by volume.

6. A lip balm composition according to claim 5, further comprising ˜0.05% to ˜0.5% Naroli oil, ˜0.05% to ˜0.5% myrrh, ˜0.05% to ˜0.5% tea tree oil, ˜0.05% to ˜1% lavender oil, ˜0.05% to ˜0.5% Blue Moroccan chamomile, ˜1% to ˜7% and vitamin E.

7. A lip balm composition according to claim 6, further comprising an ingredient selected from a group consisting of ˜0.1% to ˜8% Manuca honey and ˜0.1 to ˜8% aloe vera.

8. A lip balm composition according to claim 6, further comprising ˜0.1 to ˜8% Manuca honey and ˜0.1 to ˜8% aloe vera.

9. A lip balm composition comprising a combination of ˜10% to ˜25% bees wax, ˜5% to ˜15% cocoa butter, ˜30% to ˜50% Shea butter, and ˜20% to ˜35% Jojoba oil, ˜0.05% to ˜0.5% Naroli oil, ˜0.05% to ˜0.5% myrrh, ˜0.05% to ˜0.5% tea tree oil, ˜0.05% to ˜1% lavender oil, ˜0.05% to ˜0.5% Blue Moroccan chamomile, ˜1% to ˜7% and vitamin E, wherein all percentages are by volume.

10. A lip balm composition according to claim 8, further comprising an ingredient selected from a group consisting of ˜0.1% to ˜8% Manuca honey and ˜0.1 to ˜8% aloe vera.

11. A lip balm composition according to claim 9, further comprising ˜0.1 to ˜8% Manuca honey and ˜0.1 to ˜8% aloe vera.
